(19) United States
(12) Patent Application Publication
(10) Pub. No.: US 2008/0141384 A1
(43) Pub. Date: Jun. 12, 2008

(54) Composition For Screening Anti-Hypertension Drug Comprising Mammal Tctp Gene or Its Protein Product, and Method For Screening Anti-Hypertension Drug Using Said Composition

(75) Inventors: Kyung-Lim Lee, Seoul (KR); Goo-Taeg Oh, Daejon (KR); Myeong-Chan Cho, Cheongju (KR); Min-Jeong Kim, Kyunggi-do (KR)

Correspondence Address: ROTHWELL, FIGG, ERNST & MANBECK, P.C., 1425 K STREET, N.W., SUITE 800, WASHINGTON, DC, 20005, US

(73) Assignee: EWHA UNIVERSITY INDUSTRY COLLABORATION FOUNDATION, Seodaemoon-ku, Seoul (KR)

(21) Appl. No.: 10/561,864

(22) PCT Filed: Jun. 16, 2004
(86) PCT No.: PCT/KR04/01435

§ 371 Date: Jan. 28, 2008

(30) Foreign Application Priority Data
Jun. 21, 2003 (KR) 10-2003-0040519
Jun. 15, 2004 (KR) 10-2004-0043909

(51) Int. Cl. A01K 67/027; C07H 21/02; C07K 14/00; G01N 33/00

(52) U.S. Cl. 800/3; 536/23.1; 530/350; 436/94; 436/86; 800/18; 800/25

(57) ABSTRACT

The present invention relates to a composition for screening antihypertensive drugs, which contains a mammalian TCTP gene or a protein expressed therefrom, as well as a method for screening antihypertensive drugs using the composition. TCTP contained in the inventive composition acts as a cytoplasmic inhibitor of the Na/K APTase pump, thus causing hypertension and heart hypertrophy. Therefore, the inventive composition and the screening method using the same will be useful for the investigation and development of antihypertensive drugs. Furthermore, the inventive TCTP-overexpressing transgenic mice shows hypertension or heart hypertrophy as a phenotype. Thus, the inventive transgenic mice will be useful in screening test substances developed for the treatment and improvement of hypertension and heart hypertrophy, by administering the test substance to the transgenic mice and observing the extent of treatment and improvement.

### BACKGROUND OF THE INVENTION

1. Technical Field

The present invention relates to a composition for screening antihypertensive drugs, and a method for screening antihypertensive agents using the composition.

2. Background Art

Translationally controlled tumor protein (TCTP) is a protein known to have histamine-releasing function, as first reported by Thueson et al in a paper “J Immunol., 123(2):626-32, (1979)”. The paper describes that a histamine-releasing factor (HRF) has the activity to release histamine from cultured human mononuclear cells.

HRF which is a product from activated immune cells was defined as a substance that induce histamine release by interaction with basophils and mast cells. Then, it was classified into two categories. One requires IgE on cell surfaces upon histamine liberation, and the other acts in an IgE-independent manner. Of them, the IgE-dependent histamine-releasing factor (HRF) is believed to stimulate histamine release from basophils without antigens, thus playing an important role in the development of late allergic inflammatory responses.

In mice, the HRF protein is termed P21 meaning a 21-kDa polypeptide, and in human beings, it is known as P23. Since it was established in 1988 that the expression of HRF is regulated at the translation level by serum and regulated at the transcription level by lead, copper, cadmium, etc., HRF has been named “translationally controlled tumor protein (TCTP)”.

21 was reported to be an acidic peptide which is a 2D-PAGE reference map and has an isoelectric point of 4.9 and a molecular weight of 24 kDa. Then, by MacDonald et al. (Science, 269:688-690, 1995), HRF was isolated and purified from the lymphocytes of atopic patient and the biological fluids of allergic patient, and by its amino-terminal sequencing, its was found to be the same protein as TCTP which has been known as a growth-related protein.

TCTP has been known as a tumor-specific protein until 1980s, and its synthesis has been thought to be associated with the proliferative stage of tumors. However, it was reported that TCTP is found in all normal cells excluding kidney and renal cell carcinomas and shows high homology in almost all species.

TCTP is thus thought to be a housekeeping protein which plays a very important role in cells and is expressed at a constant level, but its definite functions are not yet known.

As disclosed in Korean Patent Application No. 10-2001-0027896, the present inventors previously found that HRF known to be the same protein as TCTP interacts with the third cytoplasmic loop of the subunits of the Na/K-ATPase pump.

TCTP is a protein obtained by performing yeast two-hybrid screens from a rat skeletal muscle library using the third cytoplasmic loop of the Na/K-ATPase pump as bait. Its interaction is not isoform specific, and it interacts only with the third cytoplasmic loop of the five cytoplasmic loops of the Na/K-ATPase pump. This interaction was confirmed again by biacore assay, coimmunoprecipitation, and confocal microscope. At this time, TCTP was shown to act as a cytoplasmic repressor of the Na/K-ATPase pump.

The Na/K-ATPase pump is a membrane protein which is present on the cellular membrane and consists of two subunits, i.e., a 112-kDa subunit and a 55-kDa subunit. The most important function of the Na/K-ATPase pump is to maintain the concentration of K⁺ ions in cells at a high level by ion transport across the membrane, and to increase the concentration of Na⁺ ions outside cells, thus regulating the intracellular Na⁺ and K⁺ ion concentrations.

Moreover, the Na/K-ATPase pump also acts as a signaling molecule in protein-protein interactions. Substances that inhibit the ion concentration regulatory function of the Na/K-ATPase pump include glycoside ouabain, digoxin, digitoxin and the like, and the long-term administration of such glycosides results in the contraction of blood vessels and the increase of blood pressure. Presumably, this is attributed to an increase in the intracellular Na⁺ and Ca²⁺ concentrations caused by a reduction in the function of the Na/K-ATPase pump.

In connection with this, a hypothesis has been proposed that a endogenous ouabain-like factor suppressing the activity of the Na/K-ATPase pump is present in cells and contributes to cause hypertension and heart hypertrophy. Recently, experimental evidence was also proposed that the repression of the Na/K-ATPase pump could activate NF-kB by cross talk with EGFR so as to cause heart hypertrophy (Haas, M., Askari, A., and Xie, Z., J. Biol. Chem., 275:27832-27837, 2000).

As a result, it is expected that inhibitors of the Na/K-ATPase pump will cause hypertension and heart hypertrophy, and effective screening of substances capable of inhibiting the activity of these inhibitors will be very important in the development of antihypertensive agents.

Accordingly, the present inventors have confirmed that TCTP causes hypertension and heart hypertrophy by inhibiting the activity of the Na/K-ATPase pump, and developed a composition and method for screening antihypertensive drugs using TCTP, thereby perfecting the present invention.

### DISCLOSURE OF THE INVENTION

An object of the present invention is to provide a composition for screening antihypertensive drugs, which contains a mammalian TCTP or a protein expressed therefrom, as well as a method for screening antihypertensive drugs using the composition.

The present invention provides a composition for screening antihypertensive drugs, which contains a mammalian TCTP gene.

The TCTP gene contained in the inventive composition is a gene found in almost all species of mammalian cells and has either a base sequence of SEQ ID NO: 1 or a base sequence having one or more disruption, deletion, insertion, point, substitution, nonsense, missense, polymorphism or rearrangement mutations in the base sequence of SEQ ID NO: 1.

In one aspect, the present invention provides a composition for screening antihypertensive drugs, which contains a mammalian TCTP protein.

The TCTP protein contained in the inventive composition is a housekeeping protein which is expressed in almost all species of mammalian cells at a constant level. This protein has an amino acid sequence of SEQ ID NO: 2, or is a polypeptide fragment which can be expressed from a gene of a base sequence having one or more disruption, deletion, insertion, point, substitution, nonsense, missense, polymorphism or rearrangement mutations in the base sequence of SEQ ID NO: 1 and shows physiological activity equal to that of TCTP.

In another aspect, the present invention provides a method for screening antihypertensive drugs, which uses the TCTP gene-containing composition as a target substance.

The present invention provides a method for screening antihypertensive drugs, the method comprising the steps of: contacting the TCTP gene-containing composition with a test substance, and examining the reaction between the composition and the test material so as to determine if the test substance exhibit the activity to inhibit the expression of the gene contained in the composition.

In another embodiment, the present invention provides a method for screening antihypertensive drugs, which uses the TCTP protein-containing composition as a target substance.

The present invention provides a method for screening antihypertensive drugs, the method comprising the steps of: contacting the TCIP protein-containing composition with a test substance; and examining the reaction between the composition and the test substance so as to determine if the test substance exhibits the activity to inhibit the function of the protein contained in the composition.

TCTP, which is a protein binding to the large cytoplasmic loop of the Na/K APTase pump, acts an intracellular inhibitor of the Na/K APTase pump (FIG. 1), thus causing hypertension and heart hypertrophy.

A mechanism by which TCTP causes hypertension and heart hypertrophy seems to be associated with a phenomenon where the inhibition of the Na/K APTase pump function influences the response and contraction of vascular smooth muscles and heart muscles so as to cause hypertension.

Mice which had been transformed to overexpress TCTP shows phenotypes of remarkable hypertension symptoms (FIG. 5) and heart hypertrophy (FIG. 6) as compared to normal mice.

Accordingly, in the present invention, antihypertensive drugs are screened using a TCTP gene or a protein expressed therefrom, as a target substance.

The inventive screening method comprises the steps of contacting the composition with a test substance, and examining the reaction between the composition and the test substance so as to determine if the test substance exhibits the activity to inhibit either the expression of the gene or the function of the protein.

In the inventive screening method, the reaction between the TCTP gene-containing composition and the test substance can be examined by conventional methods which are used in confirming that DNA-DNA, DNA-RNA, and DNA-protein reactions occurred.

Examples of the methods which can be used to examine the reaction include: a hybridization test to examine the binding between the gene and the test substance in vitro; a method of measuring the expression level of the gene by Northern blot analysis after reaction between mammalian cells and the test substance; and a method where the gene linked with a reporter gene is introduced into cells and then reacted with the test substance, and the expression level of the reporter protein is measured.

In this case, in addition to the TCTP gene, the inventive composition may contain distilled water or buffer which maintains the structure of nucleic acid stable.

In the inventive screening method, the reaction between the TCTP protein-containing composition and the test substance can be examined by conventional methods which are used in confirming that protein-protein reaction occurred.

Examples of the methods which can be used to examine the reaction include: a method of measuring the activity of the TCTP gene or protein after reacting the TCTP gene or protein with the test substance; a yeast two-hybrid method; screening of a phage-displayed peptide clone binding to the TCTP protein; high throughput screening (HTS) using natural and chemical library, drug hit HTS; cell-based screening; and screening methods using a DNA array.

In this case, in addition to the protein expressed from the TCTP gene, the inventive composition may contain buffer or reaction solution which maintains the structure or physiological activity of the protein stable. Furthermore, for in vivo experiments, the inventive composition may contain either a cell expressing the protein, or a cell containing a plasmid which expresses the protein under the presence of a promoter capable of regulating transcriptional level.

In the inventive screening method, the test substances may be individual nucleic acids, proteins, or other extracts or natural substances, which are either presumed to have the possibilities as antihypertensive drugs according to a conventional selection method or randomly selected.

Antihypertensive drug candidates obtained by the inventive screening method will act as leading compounds in a subsequent step for developing antihypertensive drugs. The structure of the leading substances can be modified and optimized such that it can exhibit an inhibitory effect against the TCTP gene or the protein expressed therefrom. This will result in the development of new antihypertensive drugs.

Since the drugs thus obtained will exhibit a partial or complete inhibitory effect against the mammalian TCTP gene or the protein expressed therefrom, they can inhibit hypertension, heart hypertrophy, and other diseases, which are caused by the TCTP gene or the protein expressed therefrom.

As a result, the inventive composition and the screening method using the same will be useful for the investigation and development of antihypertensive drugs against pathogenic bacteria including those derived from mammals.

In another aspect, the present invention provides transgenic mice which contain the mammalian TCTP gene at somatic and generative cells by the introduction of the mammalian TCTP gene at the embryonic stage, and thus show phenotypes of hypertension and heart hypertrophy by the overexpression of a TCTP protein from the TCTP gene.

In the inventive transgenic mice, the TCTP gene may have either the base sequence of SEQ ID NO: 1 or a base sequence having one or more disruption, deletion, insertion, point, substitution, nonsense, missense, polymorphism or rearrangement mutations in the base sequence of SEQ ID NO: 1.

For introduction into the embryos of the inventive transgenic mice, the TCTP gene is inserted into a transformation vector for mice. Preferably, the TCTP gene is inserted into a transformation vector DNA containing a cytomegalovirus enhancer (CMV-IE), a chicken beta-actin promoter and a rabbit beta-globin poly A-tail.

The embryos which can be used to produce the inventive transgenic mice are embryos derived from inbred or hybrid mice. Preferably, C57BL/6N inbred mice or C57BL/6J+CBA/N hybrid mice can be used.

The embryos of the inventive TCTP-overexpressing transgenic mice were deposited under accession number KCTC 10640BP on May 21, 2004 with the Korean Collection for Type Cultures (KCTC), Korean Research Institute of Bioscience and Biotechnology.

In another aspect, the present invention provides a method for producing TCTP-overexpressing transgenic mice.

The inventive method for producing TCTP-overexpressing transgenic mice comprises the steps of: 1) inserting a mammalian TCTP gene into a transformation vector to produce a recombinant gene construct for transformation; 2) microinjecting the recombinant gene construct from the step 1) into the male pronucleus of the embryo of a mouse; 3) implanting the microinjected embryo into a surrogate mother mouse; and 4) selecting TCTP-overexpressing transgenic mice from the progeny of the surrogate mother mouse, by confirming that the transgenic mice have the TCTP gene inserted into a genomic DNA, express a TCTP protein and show a phenotype of hypertension or heart hypertrophy.

In the step 1), the TCTP gene has either the base sequence of SEQ ID NO: 1 or a base sequence having one or more disruption, deletion, insertion, point, substitution, nonsense, missense, polymorphism or rearrangement mutations in the base sequence of SEQ ID NO: 1.

Moreover, the transformation vector which is used in the step 1) may be a vector for transforming mice, which is known in the art. A promoter in the vector may be either a promoter inducing expression in total tissue or a promoter inducing tissue-specific expression, and contains not only an enhancer for activating the promoter, and a poly A-tail following a terminator. Preferably, a vector containing a cytomegalovirus enhancer (CMV-IE), a chicken beta-actin promoter and a rabbit beta-globin poly A-tail may be used.

The mammalian TCTP gene is inserted into the vector for transforming mice so as to prepare a recombinant gene construct for transformation. Then, the DNA base sequence of the recombinant gene construct is analyzed, produced at large amounts, purified in various steps, and diluted in a microinjection solution to a concentration suitable for microinjection.

In the step 2), the embryos to be microinjected with the TCTP gene are prepared from inbred or hybrid mice, and the recombinant gene construct is microinjected into the male pronuclei of the prepared mouse embryos using micromanipulator. The insertion of the gene into the male pronuclei is confirmed by the expansion of the male pronucleus. The embryos microinjected with the TCTP gene are cultured to the two-cell stage in a CO₂ incubator.

In the step 3), the surrogate mother mouse to be implanted with the embryo microinjected with the foreign gene in the step 2) is prepared by mating an ICR female mouse with a vasectomized male mouse. Then, the embryo microinjected with the TCTP gene in the above step is implanted into the oviduct of the surrogate mother mouse.

In the step 4), progeny are born from the surrogate mother mouse after implantation with the embryo, and in order to confirm the introduction and expression of the TCTP gene in the born progeny, DNA and protein are extracted from the mouse tail. The inventive TCTP-overexpressing transgenic mice are selected from the progeny, by confirming that the transgenic mice have the TCTP gene inserted into the mouse genomic DNA, express a TCTP protein and show a phenotype of hypertension or heart hypertrophy.

The method for producing the inventive transgenic mice can be performed by any known method (Ian J. Jackson; Catherine M. Abbott; Mouse genetics and transgenics: a practical approach, Oxford; New York: Oxford University Press, 2000) or a modification of the known method.

In another aspect, the present invention provides a method for screening antihypertensive drugs, which comprises administering test substances to the transgenic mice overexpressing TCTP; observing the extent of improvement in hypertension and heart hypertrophy symptoms in the animal; and screening test substances showing the improvement.

### BRIEF DESCRIPTION OF DRAWINGS

FIG. 1 shows the Na/K APTase pump inhibitory activity of TCTP contained in the inventive screening composition.

FIG. 2 shows a genetic map required for the production of transgenic mice which overexpress TCTP contained in the inventive screening composition.

FIG. 3 shows that a TCTP foreign gene is inserted into a genomic DNA extracted from the tail of TCTP-overexpressing transgenic mice.

FIG. 4 shows the overexpression of a TCTP gene in the internal organs of TCTP-overexpressing transgenic mice.

FIG. 5 shows hypertension symptoms in TCTP-overexpressing transgenic mice. FIG. 5a shows measurement results for tail systolic blood pressure in the inventive transgenic mice, FIG. 5b shows measurement results for carotid systolic blood pressure in the transgenic mice, and FIG. 5c shows measurement results for left ventricular systolic blood pressure in the transgenic mice.

FIG. 6 shows the expression of heart hypertrophy in TCTP-overexpressing transgenic mice.

### BEST MODE FOR CARRYING OUT THE INVENTION

The present invention will hereinafter be described in further detail by examples. It is to be understood, however, that the scope of the present invention is not limited to or by these examples.

### Example 1

### Examination of Use of Inventive composition for Screening Antihypertensive Drugs

1) Examination of Na/K APTase Pump Inhibitory Activity of TCTP Protein Contained in Inventive Composition

The Na/K APTase pump inhibitory activity of the TCTP protein contained in the inventive composition was examined. As it was already known that TCTP interacts with the Na/K APTase pump, a test was performed to examine if the interaction has any effect on the activity of the Na/K APTase pump inhibitory activity.

RBL-2H3 cells were cultured at a suitable density and washed three times with Krebs-Ringer buffer (KRP, 140 mM NaCl, 5 mM KCl, 10 mM Na₂HPO₄, 1 mM MgSO₄, 1.4 mM CaCl₂, 2.5 mM glucose, pH 7.4). Subsequently, the cells were incubated at 37° C. for 15 minutes, and added with 0.1% BSA-containing KRP buffer containing 1 mM ouabain.

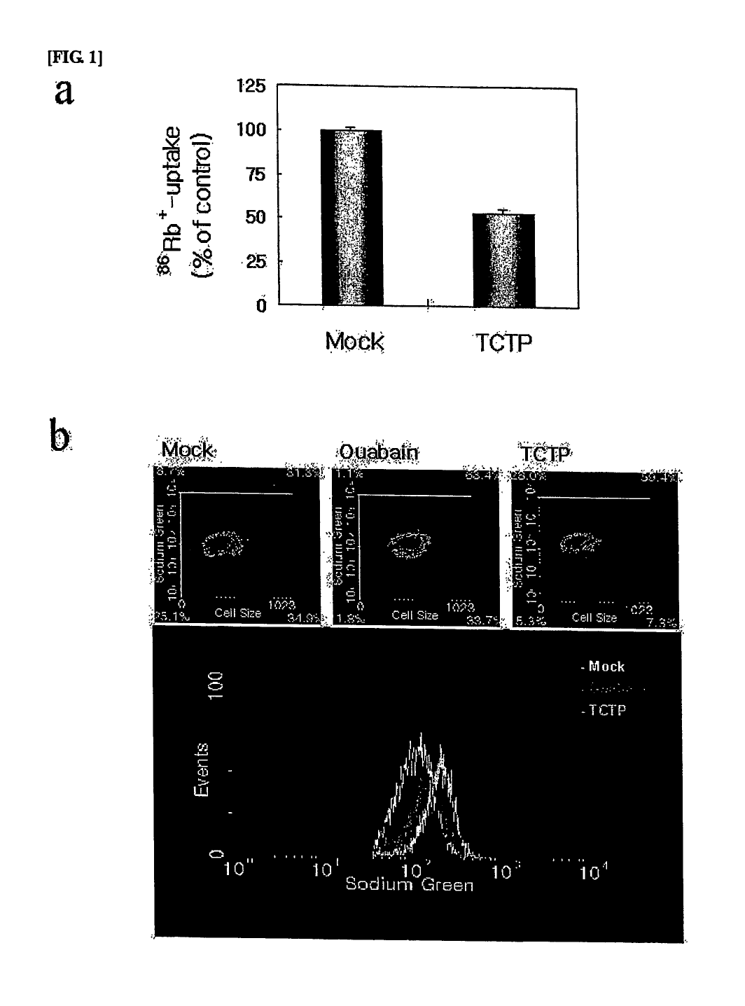

To measure the transport of K⁺ ions caused by the Na/K APTase pump, cultivation was performed for 5-10 minutes using an ⁸⁶Rb⁺(0.5 Ci/ml, NEN) tracer having high sensitivity to ouabain, and 10-20 μg/ml TCTP was added. Also, a control group to which TCTP had not been added was prepared. The uptake of ⁸⁶Rb⁺ and the consumption of K⁺ were measured and the results are shown in FIG. 1.

As shown in FIG. 1a, TCTP greatly reduced the activity of the Na/K APTase pump to 53.5%.

To confirm the above results again, FACS analysis was performed using a Na-sensitive dye.

As shown in FIG. 1b, TCTP increased the intracellular Na⁺ concentration, unlike the control group (Mock) where Na transport was smoothly made.

Accordingly, it can be found that TCTP contained in the inventive composition inhibits the ion concentration regulatory activity of the Na/K APTase pump, and is a cytoplasmic inhibitor of the Na/K APTase pump.

2) Confirmation of Hypertension and Heart Hypertrophy Caused by Inventive Composition

In order to confirm that hypertension and heart hypertrophy symptoms are caused in cells which continuously overexpress TCTP contained in the inventive composition, TCTP-overexpressing transgenic mice were constructed.

2-1) Construction of Transgenic Mice Overexpressing TCTP

The inventive TCTP-overexpressing transgenic mice were constructed from C57BL/6N inbred mice. In the construction process of the transgenic mice, animal tests, including the collection of mouse embryos, the microinjection of genes into the embryos, and the implantation of the embryos into surrogate mother mice, were performed in Macrogen, Inc., Korea.

A genetic map used in the construction of the TCTP-overexpressing transgenic mice is shown in FIG. 2. This genetic map was designed to express TCTP in the total tissue of the transgenic mice. In the genetic map, a cytomegalovirus enhancer (CMV-IE) and a chicken beta-actin promoter, which had been provided by the Animal Laboratory, Korean Research Institute of Bioscience and Biotechnology, were used.

This gene construct was microinjected into C57BL/6N inbred mice by the following procedures.

First, TCTP cDNA was inserted into a vector DNA containing the cytomegalovirus enhancer and the chicken beta-actin promoter and then confirmed by DNA base sequencing.

The DNAs were collected at large amounts using a Qiagen DNA purification kit, and treated with restriction enzyme BamHI/SalI so as to make a DNA fragment. The DNA fragment was electrophoresed on low-melting agarose gel so as to obtain about 3-Kbp DNA fragment containing an enhancer, a promoter, TCTP cDNA and a rabbit beta-globin poly A-tail. Gel containing the DNA fragment was cut and placed in high salt buffer (20 mM Tris-HCl, 1.0 M NaCl, 1.0 mM EDTA, pH 7.4). The collected DNAs were finally purified using an Elutip-D (Schleicher & Schuell) column, dialyzed in microinjection solution (10 M Tris-HCl, 0.1 mM EDTA, pH 7.4) and controlled to a concentration of about 4-6 ng/μl. Then, 50 μl of the DNA solution was placed into each well and stored at −20° C. for use.

The TCTP gene was microinjected into the male pronucleus of the mouse embryo using a micromanipulator (Leitz, Germany), and whether the gene had been injected into the nucleus was confirmed by the expansion of the male pronucleus.

A surrogate mother mouse to be implanted with the embryo microinjected with the foreign gene was prepared by the following procedures. ICR female mice showing puberty in natural conditions were mated with vasectomized male mice before one day of embryo collection. In this step, the male mouse that had not been mated for at least 3 days was mated at 1:2 with the female mice, and on the next day morning, the vaginal smear of the female mice was examined and a plugged female mouse was used as a surrogate mother mouse.

The embryos microinjected with the TCTP gene were cultured in a CO₂ incubator to the two-cell stage, and then healthy embryos were selected and implanted. First, an anesthetic (Avertin) was injected into the abdominal cavity of the surrogate mother mice at the amount of 0.5 mg/10 g body weight to achieve the general anesthesia of the mice. The abdominal wall midline of the anesthetized mice was incised about 1 cm, the ovaries and oviducts were taken out, and the embryo was implanted into each of the oviducts. After confirming that the ampulla of the oviducts slightly swollen, the oviducts and ovaries were carefully placed again into the abdominal cavity, and the muscular layer and the outer skin were sutured to each other.

In order to confirm that the progeny born from the surrogate mother mice expresses the TCTP gene, the tail of the progeny was cut off about 0.5 cm, and a genomic DNA was extracted from the cut tail using a lysis buffer containing proteinase K and subjected to PCR to determine the germline transmission of the TCTP gene. Also, a protein was extracted from the internal organs of the mice, including brains, heart, liver, spleen, kidneys and lungs, and subjected to Western blotting to examine the expression of the TCTP protein.

As a result, TCTP transgenic mice confirmed to overexpress the TCTP gene were obtained from C57BL/6N inbred mice, and the embryos of the transgenic mice were deposited under accession number KCTC 10640BP on May 21, 2004 with the Korean Collection for Type Cultures (KCTC), Korean Research Institute of Bioscience and Biotechnology.

2-2 Construction of TCTP-overexpressing Transgenic Mice

Transgenic mice were constructed in the same manner as in Example 2-1 except that C57B:/6J+CBA/N hybrid mice were used.

PCR and Western blotting were performed on the TCTP-overexpressing transgenic mice constructed from the hybrid mice in the same manner as in Example 2-1, so as to examine whether the TCTP gene was inserted or not and whether the TCTP protein was expressed or not.

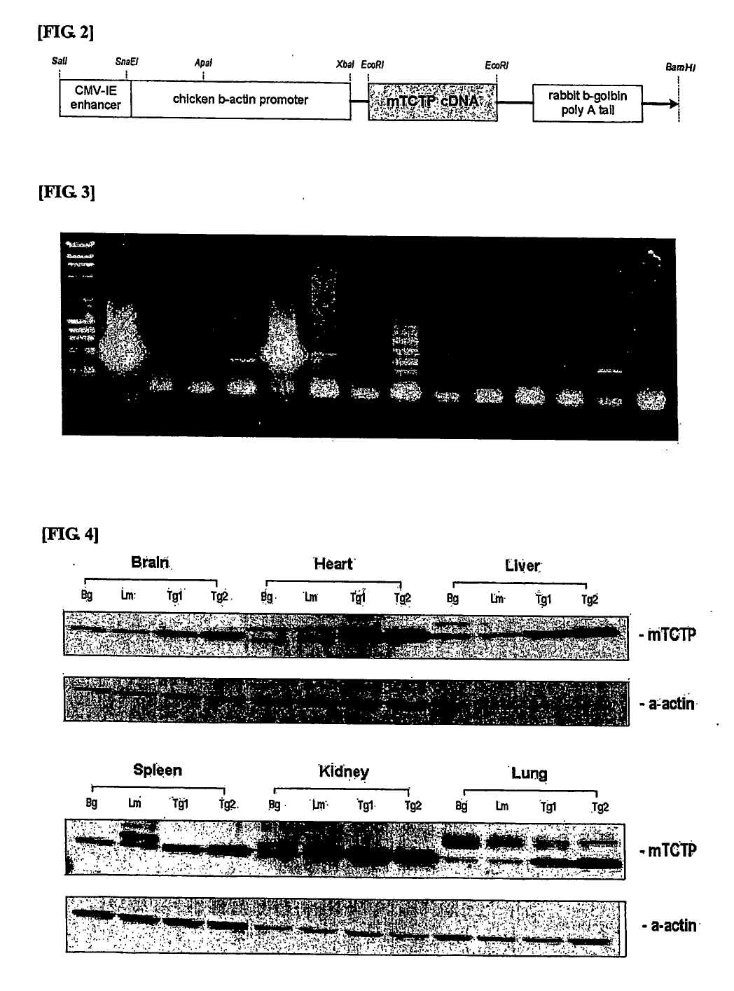

The results are shown in FIG. 3, an electrophoresis photograph. In FIG. 3, lane 2 is a positive control group, and the PCR product of the same position as that of the positive control group was observed at lane 6, suggesting that the TCTP gene was inserted into the progeny at the lane 6.

A protein was extracted from the internal organs of the mice, including brains, heart, liver, spleen, kidneys and lungs, and subjected to Western blotting. The results confirmed that the TCTP protein is expressed in all the internal organs of the transgenic mice.

2-3) Confirmation of Hypertension and Heart Hypertrophy caused in TCTP-overexpressing Transgenic Mice

On each of the male and female of the TCTP-overexpressing transgenic hybrid mice constructed in Example 2-2), the measurement of systolic blood pressure was performed by the tail-cuff method, and the measurements of carotid systolic blood pressure and left ventricular systolic blood pressure were performed by a direct method using a catheter. Also, left ventricular hypertrophy was measured to examine whether heart hypertrophy occurred or not.

The tail systolic blood pressure (T-SBP) was measured using a computerized tail-cuff system (BA-98A system, Softron Co). The blood pressure measurement on the mice was initiated from 11 weeks after the birth of the mice, and repeated at one-week intervals on the same mice. In this case, the mice were divided into four groups consisting of male and female control mouse groups, and male and female transgenic mouse groups, and at least three mice were selected from each group and measured for blood pressure.

Furthermore, on each of the male and female of 5-6-week, 9-12-week, and 19-20-week-old transgenic mice, a 1.4 French high-fidelity micromanometer catheter was inserted directly into the carotid of the mice, and the carotid systolic blood pressure (C-SBP) was measured. The catheter was pushed in the left ventricle, and the left ventricular systolic pressure (LVSP) was measured.

The left ventricular hypertrophy was determined by measuring the ratio of left ventricular weight (LVW) (mg) to body weight (BW) (g) in the 19-week-old mice.

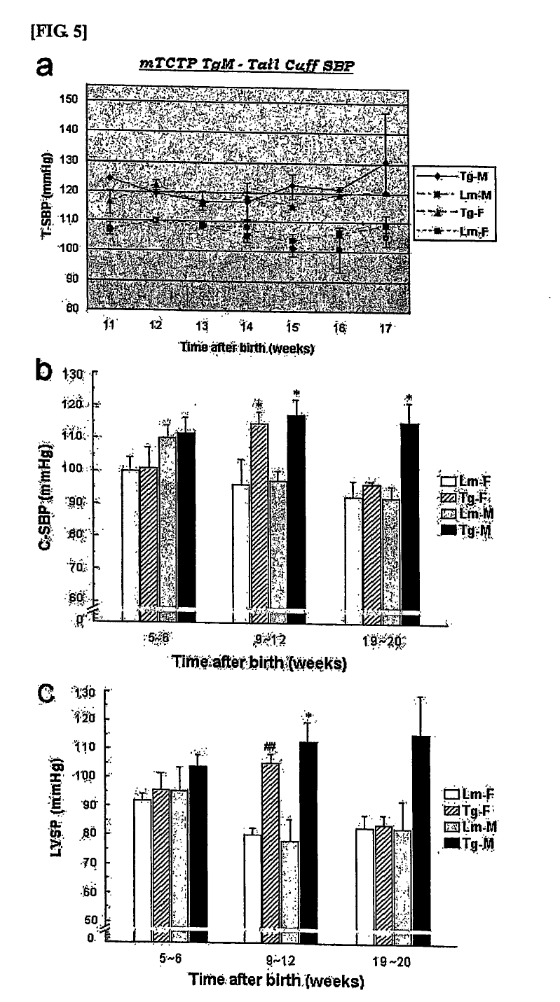

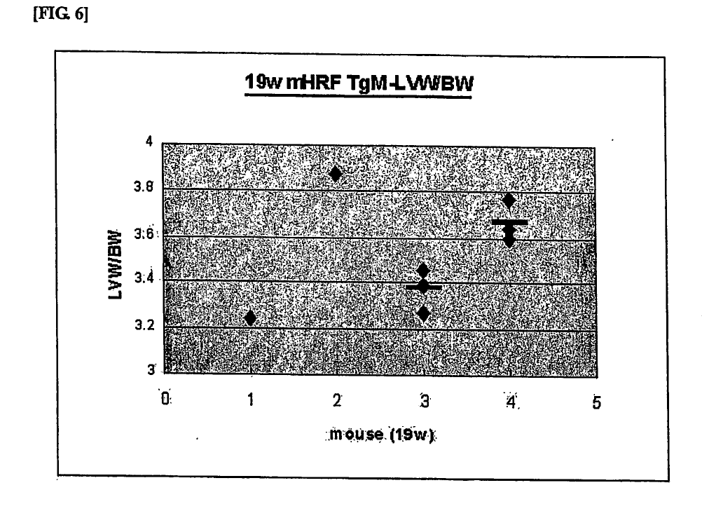

The test results are shown in FIGS. 5a to 5c and FIG. 6.

FIGS. 5a to 5c show hypertension symptoms in the TCTP-overexpressing transgenic mice. In the figures, Tg denotes transgenic mice, Lm denotes non-transgenic littermates as a control group, M denotes a male, and F denotes a female.

As shown in FIGS. 5a to 5c, the TCTP-overexpressing male and female transgenic mice showed more than 15-mmHg increases in the tail systolic blood pressure (T-SBP) as compared to the control group, indicating hypertension symptoms. Furthermore, the carotid systolic blood pressure (C-SBP) and the left ventricular systolic blood pressure (LVSP) were about 15-20 mmHg higher in the TCTP-overexpressing transgenic mice than those in the control group, and such hypertension symptoms were shown in more than 6-week-old, TCTP-overexpressing transgenic mice.

FIG. 6 shows left ventricular hypertrophy in 19-week-old transgenic mice of the present invention, in which the left ventricular hypertrophy is expressed as the ratio of left ventricular weight (LWM) (mg) to body weight (BW) (g). In FIG. 6, reference numeral 1 in the horizontal axis denotes the control female mice, 2 denotes the transgenic female mice, 3 denotes the control male mice, and 4 denotes the transgenic male mice.

As shown in FIG. 6, both the male and female of the TCTP-overexpressing transgenic mice showed heart hypertrophy as compared to the control group.

As described above, as the inventive composition causes hypertension and heart hypertrophy, antihypertensive drug candidates can be developed by screening substances which react with the inventive composition.

The inventive TCTP-overexpressing transgenic mice shows hypertension and heart hypertrophy as phenotypes, so that they will be useful in screening a test substance developed for the treatment and improvement of hypertension and heart hypertrophy, in which the screening comprises administering the test substance to the transgenic mice and observing the extent of treatment and improvement of the hypertension and heart hypertrophy.

### Industrial Applicability

The inventive composition has the effect of causing hypertension and heart hypertrophy.

Thus, the inventive composition and the screening method using the same will be useful for the investigation and development of antihypertensive drugs.

Furthermore, the inventive transgenic mice shows hypertension or heart hypertrophy as a phenotype. Thus, the transgenic mice will be useful in screening test substances developed for the treatment and improvement of hypertension and heart hypertrophy.

1. A composition for screening antihypertensive drug, which contain a mammalian TCTP gene.

2. The composition of claim 1, wherein the TCTP gene has either a base sequence of SEQ ID NO: 1 or a base sequence having one or more disruption, deletion, insertion, point, substitution, nonsense, missense, polymorphism or rearrangement mutations in the base sequence of SEQ ID NO: 1.

3. A composition for screening antihypertensive drug, which contain a mammalian TCTP protein.

4. The composition of claim 3, wherein the TCTP gene has either an amino acid sequence of SEQ ID NO: 2 or is a polypeptide fragment which can be expressed from a gene of a base sequence having one or more disruption, deletion, insertion, point, substitution, nonsense, missense, polymorphism or rearrangement mutations in the base sequence of SEQ ID NO: 1 and shows physiological activity equal to that of TCTP.

5. A method for screening antihypertensive drugs, which uses the composition of claim 1 as a target substance.

6. The method of claim 5, which comprises the steps of: contacting the composition with a test substance; and examining the reaction between the composition and the test substance so as to determine if the test substance shows the activity to inhibit the expression of the gene.

7. A method for screening antihypertensive drugs, which uses the composition of claim 3 as a target substance.

8. The method of claim 7, which comprises the steps of: contacting the composition with a test substance; and examining the reaction between the composition and the test substance so as to determine if the test substance shows the activity to inhibit the expression of the gene.

9. The method of claim 8, which uses a method of measuring the activity of the TCTP gene or protein after reacting the TCTP gene or protein with the test substance; a yeast two-hybrid method; screening of a phage-displayed peptide clone binding to the TCTP protein; high throughput screening (HTS) using natural and chemical library; drug hit HTS; cell-based screening; or a screening method using a DNA array.

10. Transgenic mice which contain mammalian TCTP gene at somatic and generative cells by the introduction of the mammalian TCTP gene at the embryonic stage, and thus show phenotypes of hypertension and heart hypertrophy by the overexpression of a TCTP protein from the TCTP gene.

11. The transgenic mice of claim 10, wherein the TCTP gene has either a base sequence of SEQ ID NO: 1 or a base sequence having one or more disruption, deletion, insertion, point, substitution, nonsense, missense, polymorphism or rearrangement mutations in the base sequence of SEQ ID NO: 1.

12. The transgenic mice of claim 11, wherein the TCTP gene is inserted into a vector DNA containing a cytomegalovirus enhancer (CMV-IE), a chicken beta-actin promoter and a rabbit beta-globin poly A-tail.

13. The transgenic mice of claim 12, wherein the embryos of the transgenic mice are deposited under accession number KCTC 10640BP.

14. A method for producing TCTP-overexpressing transgenic mice, the method comprising the steps of:
1) inserting a mammalian TCTP gene into a transformation vector to produce a recombinant gene construct for transformation;
2) microinjecting the recombinant gene construct from the step 1) into the male pronucleus of the embryo of a mouse;
3) implanting the microinjected embryo into a surrogate mother mouse; and
4) selecting TCTP-overexpressing transgenic mice from the progeny of the surrogate mother mouse, by confirming that the transgenic mice have the TCTP gene inserted into a genomic DNA, express a TCTP protein and show a phenotype of hypertension or heart hypertrophy.

15. The method of claim 14, wherein the TCTP gene has either a base sequence of SEQ ID NO: 1 or a base sequence having one or more disruption, deletion, insertion, point, substitution, nonsense, missense, polymorphism or rearrangement mutations in the base sequence of SEQ ID NO: 1.

16. A method for screening antihypertensive drugs, which comprises the steps of: administering test substances to the transgenic mice of claim 10; observing the extent of improvement of hypertension and heart hypertrophy symptoms in the animals; and screening test substances showing the improvement.


SEQUENCE LISTING (2 sequences)

SEQ ID NO: 1 - DNA, 858 bp, Homo sapiens
  mRNA (1)..(858): TCTP gene
cgctcccccctccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgttaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcaaaaaaaaaaaaaaaaaaaaaaaaaaa

SEQ ID NO: 2 - protein, 172 aa, Homo sapiens
  CHAIN (1)..(172): TCTP protein
MIIYRDLISHDEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC